(19) United States
(12) Patent Application Publication
(10) Pub. No.: US 2024/0156373 A1
(43) Pub. Date: May 16, 2024

(54) Real-Time Blood Glucose Monitoring Apparatus and Manufacturing Method Therefor

(71) Applicant: MicroTech Medical (Hangzhou) Co., Ltd., Hangzhou (CN)

(72) Inventors: Pan Zheng, Hangzhou (CN); Guodong Wang, Hangzhou (CN); Zhe Song, Hangzhou (CN); Fei Yu, Hangzhou (CN)

(21) Appl. No.: 18/283,620

(22) PCT Filed: Mar. 18, 2022
(86) PCT No.: PCT/CN2022/081702

§ 371 Date: Sep. 22, 2023

(30) Foreign Application Priority Data
Mar. 23, 2021 (CN) 202110307087.7

(51) Int. Cl. A61B 5/145

(52) U.S. Cl. CPC A61B 5/14532; A61B 2560/0214; A61B 2562/12

(57) ABSTRACT

The present invention relates to the technical field of blood glucose monitoring, and in particular to a split-type real-time blood glucose monitoring apparatus and a manufacturing method therefor. The real-time blood glucose monitoring apparatus comprises a sensor assembly and an emitter assembly. The sensor assembly comprises a first shell, and a conductive plating, a blood glucose sensor and a battery which are arranged on the first shell. The emitter assembly comprises a second shell and a circuit board in the second shell. The conductive plating is arranged on a shell wall of the first shell and is connected to the blood glucose sensor and the battery, and the conductive plating is provided with at least one first connecting structure; and the circuit board is provided with at least one second connecting structure. By means of the technical solution provided in the present invention, a sensor assembly can be further miniaturized by means of combining a conductive plating and a shell; and contacts for connection are arranged in a centralized manner, such that distribution points of the contacts for connection on the sensor assembly and the emitter assembly can be reduced, and the space occupied by a contact structure can be reduced, thereby facilitating the miniaturization of the product.

### TECHNICAL FIELD

The present invention relates to the technical field of blood glucose monitoring, and in particular to a split-type real-time blood glucose monitoring apparatus and a manufacturing method therefor.

### BACKGROUND ART

The implementation of real-time blood glucose monitoring can better control the blood glucose changes of diabetic patients. It has important guiding significance for life rules, activities, exercise, diet and rational drug use, and can help patients find problems at any time and go to the hospital in time.

A real-time blood glucose monitoring apparatus comprises a sensor assembly and an emitter assembly. The sensor assembly and the emitter assembly have a split structure; wherein the sensor assembly comprises elements such as a blood glucose sensor, monitoring needle of which is implanted subcutaneously for detecting blood glucose levels; and the emitter assembly comprises a circuit board. When the monitoring apparatus is in use, the sensor assembly is attached to the human skin, and the emitter assembly is regularly connected to the sensor assembly so as to cause the circuit in the blood glucose monitoring apparatus to form a closed-loop circuit, so that the blood glucose content data detected by the blood glucose sensor is sent to a terminal device via the emitter assembly to perform operations for doctors or users such as viewing and recording by using the terminal device.

Since the sensor assembly needs to be attached to the human skin during use, in order to reduce the influence brought by the attached sensor assembly, it is necessary to miniaturize the sensor assembly. In the existing real-time blood glucose monitoring apparatuses, in order to transmit the signals from sensors to circuit boards, nearby contact conduction is required, thus limiting the layout form or limiting the miniaturization of the volume.

### SUMMARY OF THE INVENTION

In view of this, the present invention provides a real-time blood glucose monitoring apparatus and a manufacturing method therefor. In the apparatus, a conductive plating is used as a circuit board in a sensor assembly, and a protruding connecting structure is used as a connecting contact. This setting can optimize the structure of the blood glucose monitoring apparatus, and is beneficial to the miniaturization of product.

One aspect of the present invention provides a real-time blood glucose monitoring apparatus, comprising a sensor assembly and an emitter assembly, wherein the sensor assembly comprises a first shell, and a conductive plating, a blood glucose sensor and a battery which are arranged on the first shell, and the emitter assembly comprises a second shell and a circuit board in the second shell. The conductive plating is arranged on a shell wall of the first shell, and is connected with the blood glucose sensor and the battery. The conductive plating has at least one first connecting structure. The circuit board is provided with at least one second connecting structure. The sensor assembly and the emitter assembly are clamped so that the first connecting structure and the second connecting structure are connected, and the conductive plating and the circuit board form a closed-loop monitoring circuit.

Optionally, the first connecting structure is a protruding column, on a wall of which is provided a plurality of to-be-connected terminals of the conductive plating. The second connecting structure is a connector, a first end of which is connected to the circuit board and a second end of which is provided with an inwardly-concave plug-in recess. On an inner wall of the plug-in recess are provided reeds connected with the circuit board. When the first connecting structure and the second connecting structure are connected, the protruding column is plugged into the plug-in recess, and the reeds abut against the to-be-connected terminals of the conductive plating.

Optionally, the plurality of to-be-connected terminals of the conductive plating are distributed along the wall of the protruding column.

Optionally, the conductive plating has a first connecting structure, and the circuit board is provided with a second connecting structure.

Optionally, the first shell comprises a first upper shell and a first lower shell, the edges of which are connected; the conductive plating is arranged on a surface of the first lower shell, and the protruding column is fixedly connected to the surface of the first lower shell.

Optionally, the second shell comprises a second upper shell and a second lower shell, the edges of which are connected.

Optionally, the first upper shell is provided with a first through-hole, an edge of which is provided with a first sleeve. The protruding column is arranged within and runs through the first sleeve. A first annular insertion groove is formed between an outer wall of the first sleeve and the first upper shell. The center lines of the protruding column, the first sleeve and the first annular insertion groove coincide. The second lower shell is provided with a second through-hole, an edge of which is provided with a second sleeve protruding from the outer surface of the second lower shell. The connector is arranged within and runs through the second sleeve, and there is a defined spacing between an outer wall of the connector and an inner wall of the second sleeve, thereby forming a second annular insertion groove. The center lines of the connector, the second sleeve and the second annular insertion groove coincide. When the first connecting structure and the second connecting structure are connected, the first sleeve is inserted into the second annular insertion groove, and the second sleeve is inserted into the first annular insertion groove.

Optionally, a sealing structure for blocking a gap between the first sleeve and the second sleeve is provided between the outer wall of the first sleeve and the inner wall of the second sleeve.

Optionally, the sealing structure comprises at least one sealing ring, which is sleeved on the outer wall of the first sleeve.

Optionally, an outer surface of the first lower shell is provided with medical adhesive tape, and the blood glucose sensor is arranged in a middle portion of the inner surface of the first lower shell, with a detecting end of the blood glucose sensor penetrating out of the first lower shell.

Another aspect of the present invention also provides a method for manufacturing a real-time blood glucose monitoring apparatus, comprising: plating a conductive plating on a shell of a sensor assembly, and connecting a battery and a blood glucose sensor to the conductive plating to form a circuit structure in the sensor assembly; providing a first connecting structure connected to the circuit structure, providing a second connecting structure connected to a circuit board of an emitter assembly, and communicating the first connecting structure with the second connecting structure so that the circuit structure and the circuit board are connected to form a closed-loop monitoring circuit.

Optionally, the step of providing a first connecting structure connected to the circuit structure comprises: incorporating a protruding column onto the shell of the sensor assembly or fabricating a protruding column on the shell of the sensor assembly; arranging a plurality of to-be-connected terminals of the conductive plating on side walls of the protruding column; the step of providing a second connecting structure connected with a circuit board of an emitter assembly comprises: providing a connector with a plug-in recess on the circuit board, and providing reeds having the same number as the terminals of the conductive plating on an inner wall of the plug-in recess, the reeds being connected to the circuit board; by plugging the protruding column into the plug-in recess, the to-be-connected terminals of the conductive plating abut again stand communicates with the reeds, and then the circuit structure and the circuit board are connected to form a closed-loop monitoring circuit.

Optionally, the conductive plating is plated on the shell of the sensor assembly by molded interconnection device process and/or laser direct structuring process.

According to the technical solution of the present invention, a conductive plating is arranged on the shell of the sensor assembly, and the conductive plating replaces the existing circuit board structures. By reducing the circuit board structures, the sensor assembly can be further miniaturized. Furthermore, the use of the conductive plating allows for the centralization of the contacts for connection, thereby reducing distribution points of the contacts for connection on the sensor assembly and the emitter assembly, and reducing the space occupied by a contact structure, thereby facilitating the miniaturization of the product.

### BRIEF DESCRIPTION OF THE DRAWINGS

For the purpose of illustration rather than limitation, the present invention will now be described according to its preferred embodiments, particularly with reference to the accompanying drawings, in which:

FIG. 1 is a schematic structural diagram of a real-time blood glucose monitoring apparatus according to an embodiment of the present invention;

FIG. 2 is a schematic structural diagram of a sensor assembly in the real-time blood glucose monitoring apparatus according to an embodiment of the present invention;

FIG. 3 is a schematic structural diagram of a first lower shell of the sensor assembly in the real-time blood glucose monitoring apparatus according to an embodiment of the present invention;

FIG. 4 is a partial enlarged diagram of a protruding column of the sensor assembly in the real-time blood glucose monitoring apparatus according to an embodiment of the present invention;

FIG. 5 is a schematic structural diagram of an emitter assembly in the real-time blood glucose monitoring apparatus according to an embodiment of the present invention;

FIG. 6 is a schematic structural diagram of a circuit board of the emitter assembly in the real-time blood glucose monitoring apparatus according to an embodiment of the present invention;

FIG. 7 is a structural schematic diagram of a connector of the emitter assembly in the real-time blood glucose monitoring apparatus according to an embodiment of the present invention;

FIG. 8 is a cross-sectional view of the connector of the emitter assembly in the real-time blood glucose monitoring apparatus according to an embodiment of the present invention;

FIG. 9 is a cross-sectional view of the real-time blood glucose monitoring apparatus according to an embodiment of the present invention;

FIG. 10 is another schematic structural diagram of the sensor assembly in the real-time blood glucose monitoring apparatus according to an embodiment of the present invention;

FIG. 11 is another schematic structural diagram of the emitter assembly in the real-time blood glucose monitoring apparatus according to an embodiment of the present invention;

FIG. 12 is a flow block diagram of a manufacturing method of the real-time blood glucose monitoring apparatus according to an embodiment of the present invention.

In the drawings:
1: sensor assembly; 2: emitter assembly;
11: first shell; 12: conductive plating; 13: first connecting structure; 14: first clamping structure; 111: first upper shell; 112: first lower shell; 121: to-be-connected terminals; 131: protruding column; 1111: first sleeve; 1112: first annular insertion groove; 1113: sealing ring;
21: circuit board; 22: second shell; 23: second connecting structure; 24: second clamping structure; 221: second upper shell; 222: second lower shell; 231: connector; 232: plug-in recess; 233: reed; 2221: second sleeve; 2222: second annular insertion groove.

### DETAILED DESCRIPTION OF THE INVENTION

In the embodiments of the present invention, by optimizing the structures of internal elements of a blood glucose monitoring apparatus, the volume of products can be reduced, which further facilitates the miniaturization of the products, as described in detail below.

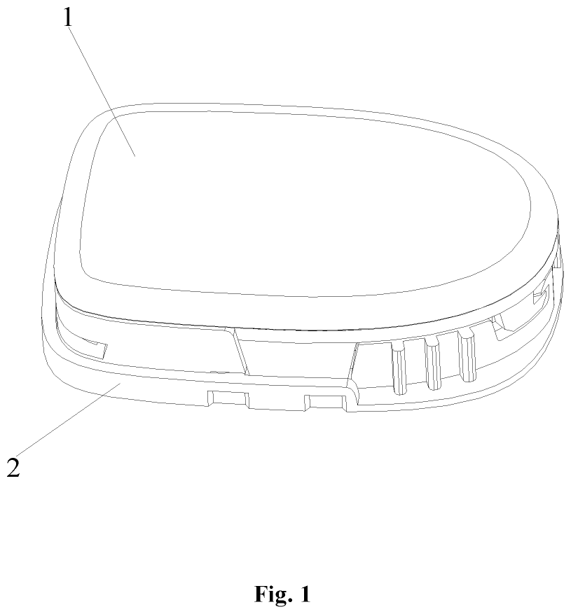

FIG. 1 is a schematic structural diagram of a real-time blood glucose monitoring apparatus according to an embodiment of the present invention. As shown in FIG. 1, the real-time blood glucose monitoring apparatus comprises a sensor assembly 1 and an emitter assembly 2, both of which are split structures. When in use, the sensor assembly 1 is used to collect blood glucose value data, which is for one-time use. The emitter assembly 2 comprises a circuit board 21. The emitter assembly 2 can be connected to the sensor assembly 1 to form a closed-loop monitoring circuit. The blood glucose value data collected by the sensor assembly 1 is sent to a terminal device via the emitter assembly 2. Since the emitter assembly 2 is not in direct contact with human skin, it can be reused. In the blood glucose monitoring apparatus, the sensor assembly 1 needs to be used for a period of time, and after the use cycle is over, the power of a battery is basically exhausted. Therefore, the battery can be arranged in the sensor assembly 1 for one-time use.

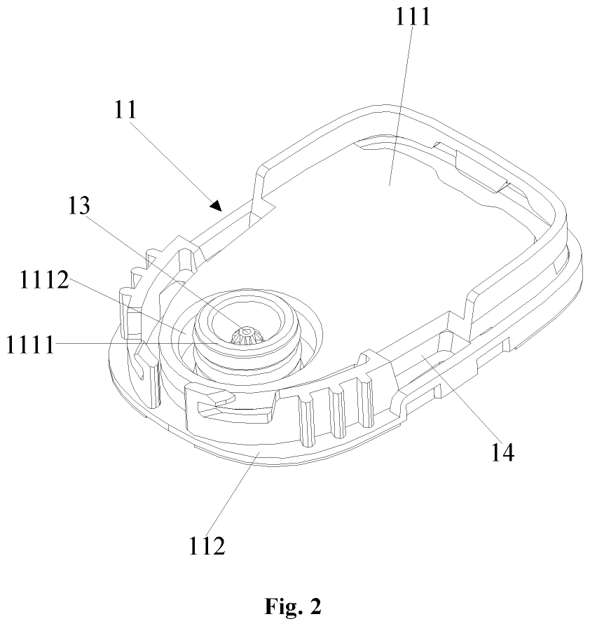

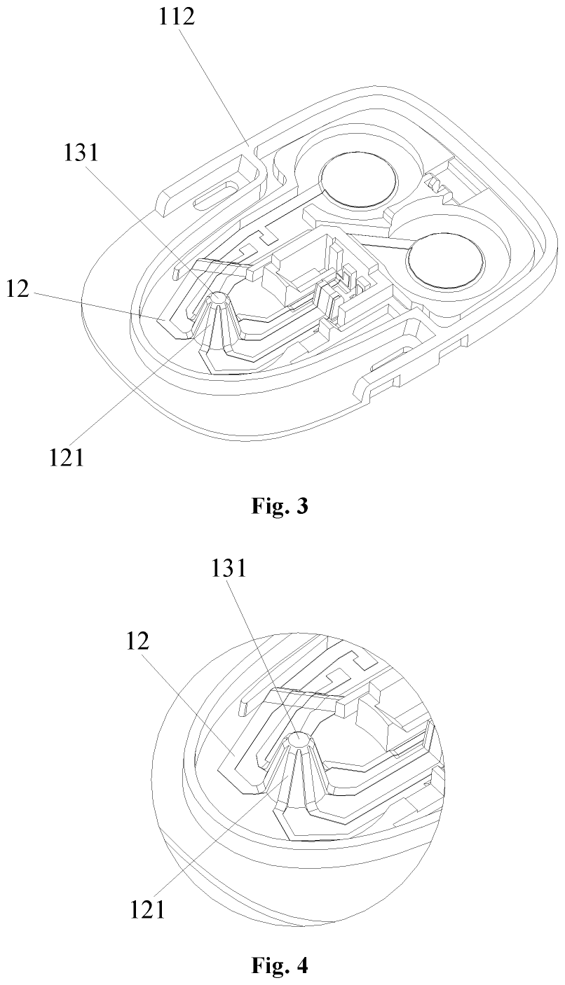

FIG. 2 is a schematic structural diagram of the sensor assembly in the real-time blood glucose monitoring apparatus according to an embodiment of the present invention; FIG. 3 is a schematic structural diagram of a first lower shell of the sensor assembly in the real-time blood glucose monitoring apparatus according to an embodiment of the present invention; and FIG. 4 is a partial enlarged diagram of a protruding column of the sensor assembly in the real-time blood glucose monitoring apparatus according to an embodiment of the present invention. As shown in FIGS. 2 to 4, the sensor assembly 1 comprises a first shell 11. The first shell 11 is provided with a conductive plating 12 connected to the first shell 11. The blood glucose sensor and the battery are both arranged in the first shell 11 and connected to the conductive plating 12. The conductive plating 12 has at least one first connecting structure 13 protruding from the first shell 11. The conductive plating 12 replaces a circuit board structure in the sensor assembly 1, and thus the space required for installing the circuit board structure in the first shell 11 can be saved, thereby reducing the volume of the sensor assembly 1. The conductive plating 12 includes a plurality of metal wires, which are plated on the first shell 11. Preferably, the metal wires are plated on the first shell 11 using a molded interconnect device (MID) process or a laser direct structuring (LDS) process. In a preferred example of an embodiment of the present invention, as shown in FIGS. 2 and 3, the first shell 11 comprises a first upper shell 111 and a first lower shell 112, the edges of which are connected. The conductive plating 12 is arranged on an inner surface of the first lower shell 112, and the protruding column 131 is fixedly connected to the inner surface of the first lower shell 112.

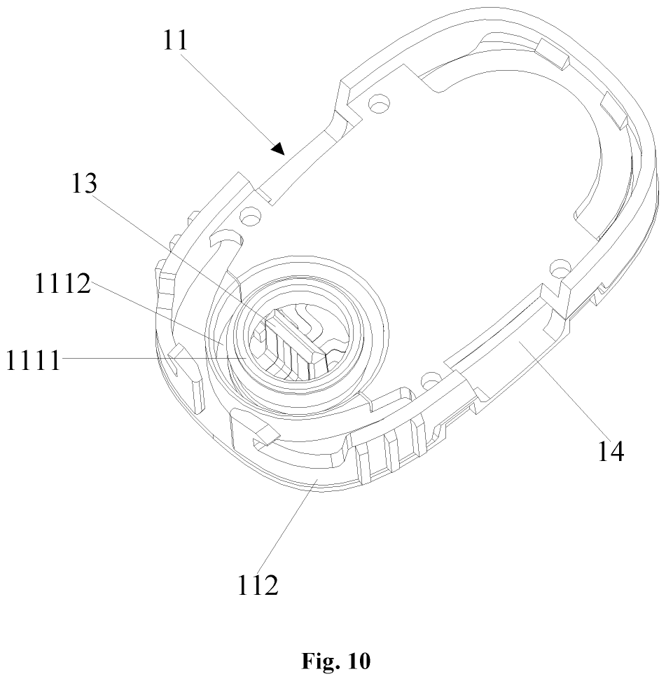

Continuing to refer to FIGS. 2 to 4, as shown in the figures, the first connecting structure 13 is a protruding column 131, which is arranged in the first shell 11, and a first end of which penetrates the first shell 11 to be located outside the first shell 11. A plurality of to-be-connected terminals 121 of the conductive plating 12 are arranged on an outer wall of the protruding column 131. In an embodiment of the present invention, the first connecting structure 13 is used as a contact for connection with the emitter assembly 2. Due to the use of the conductive plating 12 in the sensor assembly 1, when the first connecting structure 13 is provided, the to-be-connected terminals 121 of the metal wires in the conductive plating 12 can be are centrally arranged on the protruding column 131, thereby decreasing the number of contacts and achieving the purpose of further reducing the volume of the sensor assembly 1. Preferably, in the sensor assembly 1, only one first connecting structure 13 is provided. By distributing the plurality of to-be-connected terminals 121 of the conductive plating 12 along the outer wall of the protruding column 131, the to-be-connected terminals 121 can be prevented from being affected by each other. In an embodiment of the present invention, the to-be-connected terminals 121 can be arranged in various ways on the first connecting structure 13. As shown in FIG. 4, the protruding column 131 is conical form, and the plurality of to-be-connected terminals 121 are distributed circumferentially on the conical structure. FIG. 10 is another schematic structural diagram of the sensor assembly in the real-time blood glucose monitoring apparatus according to an embodiment of the present invention. As shown in FIG. 10, the protruding column 131 is in the shape of a cuboid, and the plurality of to-be-connected terminals 121 are arranged on two opposite surfaces of the protruding column 131.

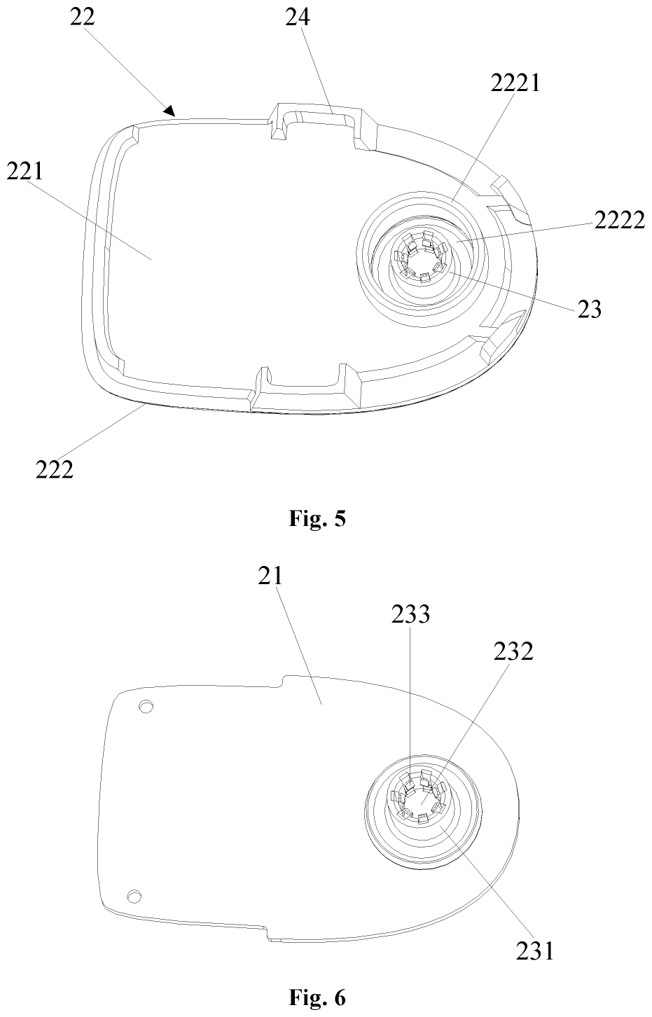

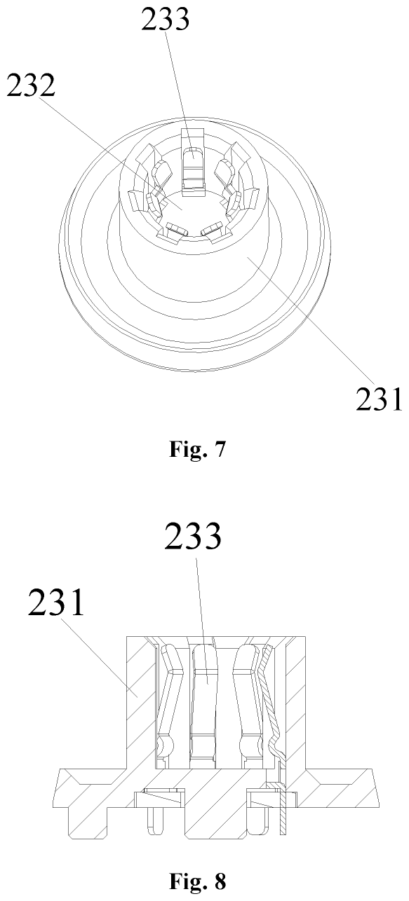

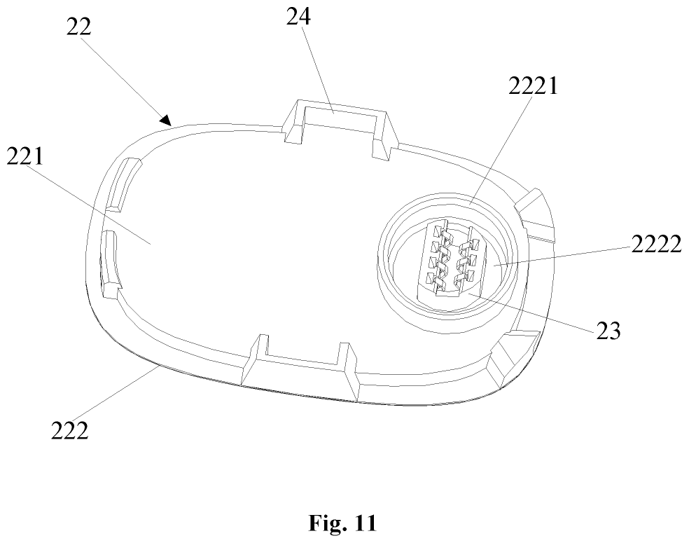

FIG. 5 is a schematic structural diagram of the emitter assembly in the real-time blood glucose monitoring apparatus according to an embodiment of the present invention; FIG. 6 is a schematic structural diagram of a circuit board of the emitter assembly in the real-time blood glucose monitoring apparatus according to an embodiment of the present invention; FIG. 7 is a structural schematic diagram of a connector of the emitter assembly in the real-time blood glucose monitoring apparatus according to an embodiment of the present invention; and FIG. 8 is a cross-sectional view of the connector of the emitter assembly in the real-time blood glucose monitoring apparatus according to an embodiment of the present invention. As shown in FIGS. 5 to 8, the emitter assembly 2 comprises a second shell 22. The second shell 22 comprises a circuit board 21, which is provided with at least one second connecting structure 23 protruding from the second shell 22. The second connecting structure 23 is a connector 231. There is no specific limitation on the shape of the connector 231 in the embodiment of the present invention. Thus, the connector 231 may be of any shape in the event of being able to ensure connection to the protruding column 131, for example, as shown in FIGS. 5 to 8, the connector 231 is a circular connector, which can be adapted to the protruding column 131 shown in FIGS. 2 to 4. FIG. 11 is another schematic structural diagram of the emitter assembly in the real-time blood glucose monitoring apparatus according to an embodiment of the present invention. As shown in FIG. 11, the connector 231 has a shape approximating to a cuboid, in a middle portion of which is provided a square-shaped plug-in recess 232 that can be adapted to the protruding column 131 shown in FIG. 10. The first end of the connector 231 is connected to the circuit board 21. The second end of the connector 231 penetrates the second shell 11 to be located outside the second shell 22, and is provided with an inwardly-concave plug-in recess 232. On an inner wall of the plug-in recess 232 are provided reeds 233 connected to the circuit board 21. The second connecting structure 23 is adapted to the first connecting structure 13, and the number of the second connecting structure 23 is the same as that of the first connecting structure 13. When the second connecting structure 23 and the first connecting structure 13 are connected, the protruding column 131 is plugged into the plug-in recess 232, and the to-be-connected terminals 121 on the outer wall of the protruding column 131 are connected to the reeds 233, so that the elements such as the battery and the blood glucose sensor in the sensor assembly 1 communicate with the circuit board 21, thereby making it possible to form a closed-loop monitoring circuit.

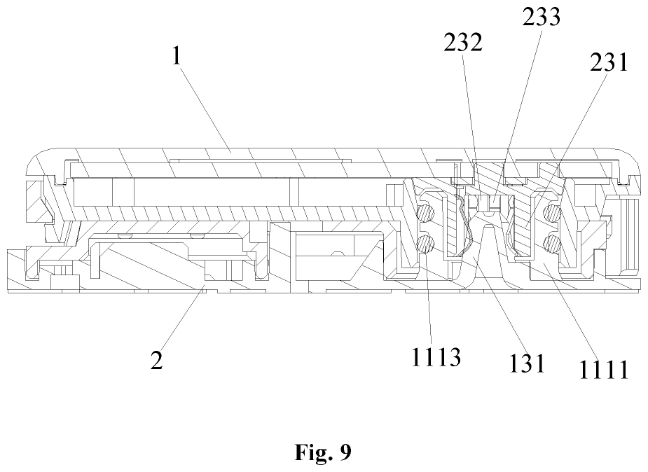

FIG. 9 is a cross-sectional view of the real-time blood glucose monitoring apparatus according to an embodiment of the present invention. As shown in FIGS. 1 to 9, the second shell 22 comprises a second upper shell 221 and a second lower shell 222, the edges of which are connected. A clamping structure is provided on an edge of the outer surface of the first upper shell 111 and an edge of the outer surface of the second lower shell 222 respectively. The clamping structures are the first clamping structure 14 and the second clamping structure 24. The outer surface of the first upper shell 111 and the outer surface of the second lower shell 222 butt together so that the clamping structures are clamped. The first shell 11 and the second shell 22 are clamped so that the first connecting structure 13 is plugged into and connected with the second connecting structure 23. In the embodiment of the present invention, the first connecting structure 13 and the second connecting structure 23 are located on the asymmetrical axis of the sensor assembly 1 and the emitter assembly 2. The outer surface of the first lower shell 112 is provided with medical adhesive tape. The blood glucose sensor is arranged in the middle portion of the inner surface of the lower shell 112, and a detection end of the blood glucose sensor penetrates the first lower shell 112 and the medical adhesive tape to be located outside the first shell 11. By adopting the setting of this position, the layout of the elements in the sensor assembly 1 can be optimized, and the sensor assembly 1 and the emitter assembly 2 can be prevented from being installed in the wrong direction, thereby facilitating the rapid installation of both.

As shown in FIGS. 2, 5 and 9, the first upper shell 111 is provided with a first through-hole, an edge of which is provided with a first sleeve 1111 protruding from the outer surface of the first upper shell 111. The protruding column 131 is arranged within and runs through the first sleeve 1111. A first annular insertion groove 1112 is formed between an outer wall of the first sleeve 1111 and the first upper shell 111. The center lines of the protruding column 131, the first sleeve 1111 and the second annular insertion groove 2222 coincide. The second lower shell 222 is provided with a second through-hole, an edge of which is provided with a second sleeve 2221 protruding from an outer surface of the second lower shell 222. The connector 231 is arranged within and runs through the second sleeve 2221, and there is a defined spacing between the outer wall of the connector 231 and the inner wall of the second sleeve 2221, thereby forming a second annular insertion groove 2222. The center lines of the connector 231, the second sleeve 2221 and the second annular insertion groove 2222 coincide. When the first connecting structure 13 and the second connecting structure 23 are connected, the first sleeve 1111 is inserted into the second annular insertion groove 2222, and the second sleeve 2221 is inserted into the first annular insertion groove 1112. By setting the structures of the first sleeve 1111 and the second sleeve 2221, the connection between the first connecting structure 13 and the second connecting structure 23 is more tightening and stable, and has a good sealing effect, when the first connecting structure 13 is plugged into the second connecting structure 23. Preferably, at least one sealing ring 1113 is sleeved on the outer wall of the first sleeve 1111. The sealing ring 1113 blocks a gap between the first sleeve 1111 and the second sleeve 2221 when the first connecting structure 13 and the second connecting structure 23 are connected. By using the sealing ring 1113, there is a better sealing effect between the first sleeve 1111 and the second sleeve 2221.

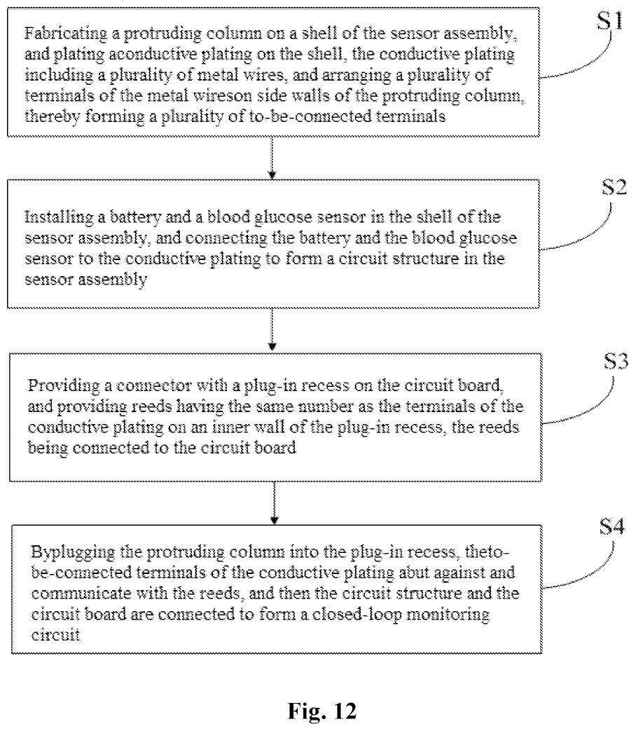

FIG. 12 is a flow block diagram of a manufacturing method of the real-time blood glucose monitoring apparatus according to an embodiment of the present invention. As shown in FIG. 12, the manufacturing process of the real-time blood glucose monitoring apparatus comprises the following steps:

Step S1: incorporating a protruding column onto the shell of the sensor assembly, for example, molding the protruding column and the shell of the sensor assembly in one piece, or fabricating the protruding column on the inner wall of the shell of the sensor assembly; and plating a conductive plating on the shell, the conductive plating including a plurality of metal wires, and arranging a plurality of terminals of the metal wires on sidewalls of the protruding column, thereby forming a plurality of to-be-connected terminals; preferably, the conductive plating is plated on the shell of the sensor assembly by molded interconnection device (MID) process and/or laser direct forming (LD) process.

Step S2: installing a battery and a blood glucose sensor in the shell of the sensor assembly, and connecting the battery and the blood glucose sensor to the conductive plating to form a circuit structure in the sensor assembly;

Step S3: providing a connector including a plug-in recess on the circuit board, and providing reeds having the same number as the terminals of the conductive plating on an inner wall of the plug-in recess, the reeds being connected to the circuit board;

Step S4: by plugging the protruding column into the plug-in recess, the to-be-connected terminals of the conductive plating abut against and communicate with the reeds, and then the circuit structure and the circuit board are connected to form a closed-loop monitoring circuit.

According to the technical solution of the embodiment of the present invention, a conductive plating is provided on the shell of the sensor assembly. The conductive plating replaces the existing circuit board structures. By reducing circuit board structures, the sensor assembly can be further miniaturized. Furthermore, by means of the conductive plating, contacts for connection can be arranged in a centralized manner, such that distribution points of the contacts for connection on the sensor assembly and the emitter assembly can be reduced, and the space occupied by a contact structure can be reduced, thereby facilitating the miniaturization of the product.

The above specific embodiments do not constitute a limitation on the scope of protection of the present invention. It should be apparent to those skilled in the art that various modifications, combinations, sub-combinations and substitutions may take place depending on design requirements and other factors. Any modifications, equivalent replacements, improvements and the like made within the spirit and principles of the present invention shall be included within the scope of protection of the present invention.

1. A real-time blood glucose monitoring apparatus, comprising a sensor assembly (1) and an emitter assembly (2), characterized in that
the sensor assembly (1) comprises a first shell (11), and a conductive plating (12), a blood glucose sensor and a battery which are arranged on the first shell (11), and the emitter assembly (2) comprises a second shell (22) and a circuit board (21) in the second shell (22);
the conductive plating (12) is arranged on a shell wall of the first shell (11) and is connected with the blood glucose sensor and the battery, the conductive plating (12) having at least one first connecting structure (13);
the circuit board (21) is provided with at least one second connecting structure (23);
the sensor assembly (1) and the emitter assembly (2) are clamped so that the first connecting structure (13) and the second connecting structure (23) are connected, and the conductive plating (12) and the circuit board (21) form a closed-loop monitoring circuit.

2. The real-time blood glucose monitoring apparatus according to claim 1, characterized in that
the first connecting structure (13) is a protruding column (131), and a plurality of to-be-connected terminals (121) of the conductive plating (12) are provided on a wall of the protruding column (131);
the second connecting structure (23) is a connector (231), a first end of which is connected with the circuit board (21) and a second end of which is provided with an inwardly-concave plug-in recess (232), and reeds (233) connected to the circuit board (21) are provided on an inner wall of the plug-in recess (232);
when the first connecting structure (13) and the second connecting structure (23) are connected, the protruding column (131) is plugged into the plug-in recess (232), and the reeds (233) abut against the to-be-connected terminals (121) of the conductive plating (12).

3. The real-time blood glucose monitoring apparatus according to claim 2, characterized in that the plurality of to-be-connected terminals (121) of the conductive plating (12) are distributed along the wall of the protruding column (131).

4. The real-time blood glucose monitoring apparatus according to claim 1, characterized in that the conductive plating (12) has a first connecting structure (13), and the circuit board (21) is provided with a second connecting structure (23).

5. The real-time blood glucose monitoring apparatus according to claim 1, characterized in that the first shell (11) comprises a first upper shell (111) and a first lower shell (112), the edges of which are connected; the conductive plating (12) is arranged on a surface of the first lower shell (112), and the protruding column (131) is fixedly connected to the surface of the first lower shell (112).

6. The real-time blood glucose monitoring apparatus according to claim 5, characterized in that the second shell (22) comprises a second upper shell (221) and a second lower shell (222), the edges of which are connected.

7. The real-time blood glucose monitoring apparatus according to claim 6, characterized in that
the first upper shell (111) is provided with a first through-hole, an edge of which is provided with a first sleeve (1111); the protruding column (131) is arranged within and runs through the first sleeve (1111); a first annular insertion groove (1112) is formed between an outer wall of the first sleeve (1111) and the first upper shell (111); and the center lines of the protruding column (131), the first sleeve (1111) and the first annular insertion groove (1112) coincide;
the second lower shell (222) is provided with a second through-hole, an edge of which is provided with a second sleeve (2221) protruding from an outer surface of the second lower shell (222); the connector (231) is arranged within and runs through the second sleeve (2221), and there is a defined spacing between an outer wall of the connector (231) and an inner wall of the second sleeve (2221), thereby forming a second annular insertion groove (2222); the center lines of the connector (231), the second sleeve (2221) and the second annular insertion groove (2222) coincide;
when the first connecting structure (13) and the second connecting structure (23) are connected, the first sleeve (1111) is inserted into the second annular insertion groove (2222), and the second sleeve (2221) is inserted into the first annular insertion groove (1112).

8. The real-time blood glucose monitoring apparatus according to claim 7, characterized in that a sealing structure for blocking a gap between the first sleeve (1111) and the second sleeve (2221) is provided between the outer wall of the first sleeve (1111) and the inner wall of the second sleeve (2221).

9. The real-time blood glucose monitoring apparatus according to claim 8, characterized in that the sealing structure comprises at least one sealing ring (1113), which is sleeved on the outer wall of the first sleeve (1111).

10. The real-time blood glucose monitoring apparatus according to claim 6, characterized in that an outer surface of the first lower shell (112) is provided with medical adhesive tape, and the blood glucose sensor is arranged in a middle portion of an inner surface of the first lower shell (112), with a detecting end of the blood glucose sensor penetrating out of the first lower shell (112).

11. A method for manufacturing a real-time blood glucose monitoring apparatus, characterized by comprising:
plating conductive plating on a shell of a sensor assembly, and connecting a battery and a blood glucose sensor to the conductive plating to form a circuit structure in the sensor assembly;
providing a first connecting structure connected to the circuit structure, providing a second connecting structure connected to a circuit board of an emitter assembly, and communicating the first connecting structure with the second connecting structure so that the circuit structure and the circuit board are connected to form a closed-loop monitoring circuit.

12. The method according to claim 11, characterized in that the step of providing a first connecting structure connected to the circuit structure comprises:
incorporating a protruding column onto the shell of the sensor assembly or fabricating a protruding column on the shell of the sensor assembly; arranging a plurality of to-be-connected terminals of the conductive plating on side walls of the protruding column;
the step of providing a second connecting structure connected with a circuit board of an emitter assembly comprises: providing a connector with a plug-in recess on the circuit board, and providing reeds having the same number as the terminals of the conductive plating on an inner wall of the plug-in recess, the reeds being connected to the circuit board;
by plugging the protruding column into the plug-in recess, the to-be-connected terminals of the conductive plating abut against and communicates with the reeds, and then the circuit structure and the circuit board are connected to form a closed-loop monitoring circuit.

13. The method according to claim 11, characterized in that the conductive plating is plated on the shell of the sensor assembly by molded interconnection device process and/or laser direct structuring process.
